(19) United States
(12) Patent Application Publication
(10) Pub. No.: US 2024/0102098 A1
(43) Pub. Date: Mar. 28, 2024

(54) BIOMARKERS OF MUSCULOSKELETAL INJURY

(71) Applicant: MARKER HOLDINGS AG, Zug 6300 (CH)

(72) Inventors: Valentina DI PIETRO, Birmingham (GB); Valentina DI PIETRO, East Wellow (GB)

(21) Appl. No.: 18/479,643

(22) Filed: Oct. 2, 2023

Related U.S. Application Data
(63) Continuation of application No. PCT/IB2022/053127, filed on Apr. 4, 2022.
(60) Provisional application No. 63/170,438, filed on Apr. 2, 2021.

(51) Int. Cl. C12Q 1/6883

(52) U.S. Cl. CPC C12Q 1/6883; C12Q 2600/118; C12Q 2600/158; C12Q 2600/178

(57) ABSTRACT

Methods of diagnosing, monitoring, treating, and predicting the course of musculoskeletal injury include determining a level of at least one RNA biomarker (e.g., miRNA) in a body fluid sample from a subject. Also described are sensor elements, detection systems, compositions, and kits for diagnosing, monitoring, treating, and predicting the course of a musculoskeletal injury.

### CROSS-REFERENCE TO RELATED APPLICATIONS

This application is a continuation of PCT Patent Application No. PCT/IB2022/053127 filed Apr. 4, 2022, which claims priority benefit of U.S. Provisional Patent Application Ser. No. 63/170,438, filed Apr. 2, 2021, the disclosure of each is incorporated by reference herein in their entirety.

### REFERENCE TO AN ELECTRONIC SEQUENCE LISTING

The contents of the electronic sequence listing (20231218_SequenceListing_ST26_11241003US1.xml; Size: 42,597 bytes; and Date of Creation: Dec. 18, 2023) is herein incorporated by reference in its entirety.

### FIELD

The present disclosure relates to compositions, kits, systems and methods for diagnosing and/or monitoring musculoskeletal injury. More particularly, the present disclosure relates to the diagnosis and monitoring of musculoskeletal injuries using RNA biomarkers.

### BACKGROUND

Musculoskeletal injury refers to damage of muscular or skeletal systems, which is usually due to a strenuous activity. In one study, roughly 25% of approximately 6300 adults received a musculoskeletal injury of some sort within 12 months—of which 83% were activity-related. Musculoskeletal injury spans into a large variety of medical specialties including orthopedic surgery (with diseases such as arthritis requiring surgery), sports medicine, emergency medicine (acute presentations of joint and muscular pain) and rheumatology (in rheumatological diseases that affect joints such as rheumatoid arthritis).

Musculoskeletal injuries can affect any part of the human body including; bones, joints, cartilages, ligaments, tendons, muscles, and other soft tissues. Symptoms include mild to severe aches, low back pain, numbness, tingling, atrophy and weakness. These injuries are a result of repetitive motions and actions over a period of time. Tendons connect muscle to bone whereas ligaments connect bone to bone. Tendons and ligaments play an active role in maintain joint stability and controls the limits of joint movements, once injured tendons and ligaments detrimentally impact motor functions. Continuous exercise or movement of a musculoskeletal injury can result in chronic inflammation with progression to permanent damage or disability.

In many cases, during the healing period after a musculoskeletal injury, a period in which the healing area will be completely immobile, a cast-induced muscle atrophy can occur. Routine sessions of physiotherapy after the cast is removed can help return strength in limp muscles or tendons. Alternately, there exist different methods of electrical stimulation of the immobile muscles which can be induced by a device placed underneath a cast, helping prevent atrophies. Preventative measures include correcting or modifying one's postures and avoiding awkward and abrupt movements. It is beneficial to rest post injury to prevent aggravation of the injury.

There are three stages of progressing from a musculoskeletal injury: (i) cause; (ii) disability; and (iii) decision. The first stage arises from the injury itself whether it be overexertion, fatigue or muscle degradation. The second stage involves how the individual's ability is detrimentally affected as disability affects both physical and cognitive functions of an individual. The final stage, decision, is the individual's decision to return to work post recovery as musculoskeletal injuries compromise movement and physical ability which ultimately degrades one's professional career.

Despite many studies, there is a need for more sophisticated methods of diagnosing musculoskeletal injuries.

The present disclosure is directed to overcoming these and other defiiencies in the art.

### SUMMARY

The present disclosure provides methods and detection systems relating to diagnosing, monitoring, and treating musculoskeletal injuries in a human subject who has suffered such an injury by detecting one or more miRNA molecules in a biological sample from the subject. In the context of the following disclosure, the terms “level” and “amount” in reference to the level or amount of an miRNA molecule or molecules in a biological sample are used interchangeably. Furthermore, as used herein, the term “biological sample” can be used interchangeably with the term “body fluid sample.” More specifically, as used herein, the term “body fluid sample” can include, without limitation, blood, saliva, urine, and cerebrospinal fluid (CSF). As used herein, the term “musculoskeletal injury” can include, without limitation, trauma to bones, muscles, cartilage, ligaments, and/or tendons.

In one aspect, the disclosure relates to a method of diagnosing and treating a musculoskeletal injury in a human subject in need thereof. The method involves: obtaining a body fluid sample from the subject; contacting the body fluid sample with a probe comprising a nucleic acid able to bind to at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put- miR-465, put-miR-6, and Y_RNA.245, or any combination thereof; and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR-142-3p, miR-142-5p, put-miR-188, SNORD3B-2, AND U6.168, or any combination thereof determining an amount of the at least one RNA biomarker in the body fluid sample; identifying the subject as having a musculoskeletal injury where the amount of the at least one RNA biomarker is increased or decreased relative to a predetermined threshold value or relative to the amount of the RNA biomarker in a control sample; and treating the subject identified as having a musculoskeletal injury.

In another aspect, the present disclosure relates to a method of diagnosing and/or monitoring a musculoskeletal injury in a subject. This method involves: determining a level of at least one RNA biomarker in a body fluid sample obtained from the subject, wherein the at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR-142-3p, miR-142-5p, put-miR-188, SNORD3B-2, AND U6.168, or any combination thereof.

In another aspect, the present disclosure relates to a sensor element for a detection system for diagnosing and/or monitoring a musculoskeletal injury, the sensor element comprising a substrate functionalized with a probe specific for at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof; and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR- 142-3p, miR-142-5p, put-miR-188, SNORD3B-2, AND U6.168, or any combination thereof.

In another aspect, the present disclosure relates to a detection system for diagnosing and/or monitoring a musculoskeletal injury, comprising: a sensor element according to the present disclosure, and a detection device capable of detecting the binding of a target RNA biomarker to the probe.

In another aspect, the present disclosure relates to a method for determining a course of action for a subject suspected of having a musculoskeletal injury, comprising applying a body fluid sample obtained from the subject to a detection system according to the present disclosure, and if an upregulated level or a downregulated level of the at least one RNA biomarker is detected, providing a treatment for the musculoskeletal injury.

In another aspect, the present disclosure relates to a method of treating a subject with suspected of having a musculoskeletal injury. This method involves: determining whether an upregulated level or a downregulated level of at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof; and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR- 142-3p, miR-142-5p, put-miR-188, SNORD3B-2, AND U6.168, or any combination thereof, is detectable in a body fluid sample obtained from the subject, and if an upregulated level or a downregulated level of at least one RNA biomarker is detected, providing treatment for the musculoskeletal injury to the subject.

In another aspect, the present disclosure relates to a method of detecting an RNA biomarker in a body fluid sample. This method involves: obtaining a body fluid sample from a human subject, contacting the body fluid sample with at least one oligonucleotide primer complementary to at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR-142-3p, miR-142-5p, put-miR-188, SNORD3B-2, AND U6.168, or any combination thereof amplifying the at least one RNA biomarker using a polymerase chain reaction; and detecting the amplified RNA biomarker.

In another aspect, the present disclosure relates to a kit for use in a method of diagnosing and/or monitoring a musculoskeletal injury in a body fluid sample from a human subject, the kit comprising at least one probe specific for at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof; and/or (b) selected from the group consisting of let-7a-5p, let-7f-5p, miR-135b-5p, miR-21-5p, miR- 425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR-142-3p, miR- 142-5p, put-miR-188, SNORD3B-2, AND U6.168, or any combination thereof.

In another aspect, the present disclosure relates to a composition for use in a method of diagnosing and/or monitoring a musculoskeletal injury in a body fluid sample from a human subject, the composition comprising at least one probe specific for at least one RNA biomarker: (a) selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof; and/or (b) selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR- 142-3p, miR-142-5p, put-miR-188, SNORD3B-2, AND U6.168, or any combination thereof.

### DETAILED DESCRIPTION

The present disclosure provides methods of diagnosing or monitoring musculoskeletal injury in a subject.

As used herein, the term “musculoskeletal injury” refers to, without limitation, trauma to bones, muscles, cartilage, ligaments, and/or tendons.

As used herein, the term “musculoskeletal injury biomarker” refers to RNA biomarker found to be associated with muscuolskeletal injuries in animals and humans. The musculoskeletal injury biomarkers can be RNA biomarkers that are found in body fluids of an Animal or human, including, without limitation, body fluids such as blood, saliva, urine, or cerebrospinal fluid (CSF). Specific examples of musculoskeletal injury biomarkers in accordance with the present disclosure include, without limitation, those RNA biomarkers found in Table A.

MicroRNAs (miRNAs) are an abundant class of highly conserved, non-coding RNA molecules of approximately 22 nucleotides in length that induce mRNA degradation, translational repression or both via pairing with partially complementary sites in the 3′UTR of target genes. The human genome encodes over 2,000 miRNAs, which may target about 60% of all genes. However, despite the abundance of miRNAs, their biomolecular functions and involvement in pathology remain to be fully elucidated. They play a central role in many biological processes including cell cycle, cell metabolism, apoptosis and immune responses, and are attracting increasing interest in clinical research as potential biomarkers for the detection, identification and classification of cancers and other disease states including neurodegenerative diseases.

For the avoidance of doubt, it will be understood that “the at least one miRNA is selected from a group of miRNAs”, as used herein, means that the method in question, whether carried out for a diagnostic, prognostic, or therapeutic purpose, can be carried out with any one of the listed miRNAs or with any plurality of the listed miRNAs (e.g., two, three, four, or more of the listed miRNAs). It follows that any one or more of the listed miRNAs may be explicitly excluded.

In some embodiments, the subject is human.

The level of the miRNA or of each miRNA in the sample may be determined quantitatively or semi-quantitatively. By “quantitatively”, it will be understood that the absolute amount or concentration of the miRNA or of each miRNA in the sample is determined. The absolute amount of the miRNA or of each miRNA in the sample can then be compared to a predetermined threshold (e.g. a published literature value for expected normal levels), a known level of the same or a reference miRNA in a control sample taken from a healthy subject, or the amount of a reference miRNA in the sample taken from the subject. In some embodiments, the subject is diagnosed as having a musculoskeletal injury when the level of the miRNA is below the predetermined threshold, or decreased relative to a reference or control sample. In other embodiments, the subject is diagnosed as having a musculoskeletal injury when the level of the miRNA is increased compared to the predetermined threshold.

By “semi-quantitatively”, it will be understood that the level of the or each miRNA of interest is measured relative to a reference.

The reference may be an invariant miRNA, i.e. a miRNA having an expression level that remains substantially unchanged between healthy subjects and those having a musculoskeletal injury. A subject may be diagnosed as suffering from a musculoskeletal injury if the level of the miRNA or of each miRNA of interest is increased or decreased relative to that of an invariant miRNA.

In some embodiments, the level of the miRNA or of each miRNA in the sample obtained from the subject may be about 0.01 times to about 100 times, about 0.05 times to about 50 times, about 0.1 times to about 10 times, about 0.5 times to about 5 times, about 1.0 to about 3 times, or about 1.5 times to about 2.0 times lower or higher than the level in the control sample, the reference level or the published value.

Where a device or method is employed to generate a value, we may qualify that value with the term “about” in order to capture the stated value and any variation of that value inherent to the device or method employed. Where values or ranges of values are specifically disclosed, “about” may mean plus-or-minus 10% of the stated value or range. For example, about 10 minutes may mean 9-11 minutes.

The level of the miRNA or of each miRNA of interest can be determined using methods known to those skilled in the art. In some embodiments, determining the level of the miRNA or of each miRNA of interest comprises amplifying the miRNA. In some embodiments, total miRNA may be first isolated from the sample using standard techniques, for example using the miRNeasy mini kit (Qiagen). The amount of the miRNA of interest can then be determined. In some embodiments, the level of the miRNA or of each miRNA of interest in the sample is determined using PCR (polymerase chain reaction). For example, quantitative PCR may be used for quantitative determination of the level of the miRNA or of each miRNA of interest. PCR may also be used for semi-quantitative determination, by comparing the level of the miRNA or of each miRNA of interest in the sample with that of a reference (e.g. an invariant miRNA).

Suitable techniques for miRNA detection and/or quantification, which will be known to those skilled in the art, include qPCR, miRNA assays, next-generation sequencing (NGS), and multiplex miRNA profiling assays.

In some embodiments, the level of the miRNA or of each miRNA of interest is determined using in-situ hybridization, for example using a probe (e.g., a labelled probe) specific for the miRNA.

The level of miRNA may be determined in a sample which was obtained from the subject immediately after injury (i.e. less than 1 hour after injury), and/or in a sample obtained at one or more time points a few hours or days after injury. Thus, changes in the miRNA level can be detected over time to enable monitoring of a musculoskeletal injury. In the event miRNA levels change over time, the methods described herein for monitoring musculoskeletal injury can be expanded to include maintaining or adjusting the subject's treatment regimen accordingly.

Depending on the specific miRNA and the type of musculoskeletal injury, the level of miRNA in the subject may change significantly over time. In some embodiments, it may therefore be advantageous to measure the miRNA relatively soon after injury to enable an accurate diagnosis. In some embodiments, the level of miRNA is determined in a sample obtained from the subject no more than 72 hours, no more than 48 hours, no more than 36 hours, no more than 24 hours, no more than 12 hours, no more than 6 hours, no more than 4 hours, no more than 2 hours or no more than 1 hour after injury.

The level of some miRNAs is substantially stable over time, thus allowing a diagnosis to be made a few hours, days or even weeks after injury. In some embodiments, the level of miRNA is determined in a sample obtained from the subject up to 20, 18, 15, 12, 10, 8, 5 or 2 days from injury.

In some embodiments, the level of miRNA is determined in a sample obtained from the subject immediately after injury (e.g. at T=0 h), at 4-12 hours after injury, at 48-72 hours after injury, or at 15 days after injury.

In some embodiments, the level of miRNA is determined in a sample obtained from the subject at least 24 hours after injury. In some embodiments, the level of miRNA is determined in a sample obtained from the subject 15 days or fewer after injury. In some embodiments, the level of miRNA is determined in a sample obtained from the subject between 24 hours and 15 days after injury, or between 24 hours and 10 days after injury, or between 24 hours and 7 days after injury, or between 48 hours and 5 days after injury.

Conveniently the sample may be any appropriate fluid or tissue sample obtained from the subject. For example, the biological sample may comprise at least one of the group consisting of: urine, saliva, whole blood, plasma, serum, sputum, semen, faeces, a nasal swab, tears, a vaginal swab, a rectal swab, a cervical smear, a tissue biopsy, and a urethral swab. In some embodiments the sample is a fluid sample. Suitably, the sample is one that can be readily obtained from the individual, such as urine, saliva, blood and sputum. In some embodiments, the sample comprises saliva, blood, plasma or serum. It will be appreciated that in some embodiments the process of obtaining the sample does not form part of the invention described herein.

In some embodiments, the sample comprises or is constituted by serum. Not only does serum have practical advantages, but it is also free of anticoagulants such as heparin, a potential inhibitor of PCR reactions. Serum may also be less affected by haemolysis, compared to plasma.

In some embodiments, the sample is saliva. Saliva can be easily obtained from the patient (e.g. pitch-side, or in the field), without specialist training or medical equipment.

The diagnosis of a subject as suffering from a musculoskeletal injury may facilitate in the determination of an appropriate treatment. The present invention thus provides a test that enables healthcare workers, such as physicians, clinicians, paramedics, and even non-medical personnel (e.g. teachers, sports coaches, military personnel) to decide on appropriate action for a subject suspected of having a musculoskeletal injury. A subject determined as having a musculoskeletal injury may therefore receive the most appropriate treatment as a result of a diagnosis being made. The method of the invention may thus further comprise directing appropriate therapy to a subject diagnosed with a musculoskeletal injury.

A subject diagnosed with a musculoskelatal outside a hospital environment, for example, at a sporting event, during combat or during play, may be removed from play or combat immediately. The subject may subsequently be started on a graduated return to play or combat.

In a further aspect, there is provided a method for determining whether it is appropriate to administer to a subject a therapy for alleviating muscolskeletal injury, the method comprising: determining a level of at least one miRNA in a sample from the subject; and determining whether or not it is appropriate to administer a therapy for alleviating muscolskeletal injury, based on the level of the at least one miRNA.

It will be appreciated that the step of administering the therapy to the subject does not form a part of the claimed method, unless specifically stated.

In some embodiments the method may further comprise administering to the subject an appropriate treatment. In some embodiments, the treatment may comprise a therapy for alleviating muscolskeletal injury. Accordingly, the invention features methods of diagnosing and treating muscolskeletal injury in a subject, the method comprising the steps of (a) obtaining a sample (e.g., a sample of blood, plasma, urine, or saliva) from the subject; (b) detecting one or more miRNAs (selected from those described herein); diagnosing the patient as having a muscolskeletal injury when the level(s) of the miRNA(s) differ from a reference standard (as described herein); and administering a treatment for the muscolskeletal injury.

In a further aspect, the invention provides a method of determining an appropriate treatment to a subject suspected of suffering from a muscolskeletal injury, the method comprising identifying whether or not the subject has a muscolskeletal injury by determining a level of at least one miRNA in a sample from the subject.

If a subject is identified as having a musculoskeletal injury, an appropriate course may include one or more of the following: further evaluating the subject, for example by further tests (e.g., medical imaging by x-ray, CT scan, or MRI, measuring muscle enzymes such as creatine kinase, SGOT, SGPT, and LDH; checking blood and urine myoglobin to diagnose and monitor possible rhabdomyolysis; and/or performing electromyography or electroneurography); removing the subject from activity (e.g., the activity during which the musculoskeletal injury was incurred); admitting the subject to hospital or a specialist clinic; and administering a therapy for alleviating the musculoskeletal injury to the subject, such as cold or heat application, analgesics such as nonsteroidal anti-inflammatory drugs (NSAIDs) or narcotics (opiates), dialysis for rhabdomyolysis, muscle relaxants (antispasmodics), immobilization, surgery (e.g., for repair of a fracture or ruptured tendon or ligament); and/or physical therapy.

In some embodiments, the subject may be subsequently monitored to track their recovery, for example in a hospital or clinic setting.

According to a further aspect of the invention, there is provided a method of detecting and/or determining a level of a target miRNA in a subject, the method comprising the steps of (a) obtaining a sample from the subject; and (b) detecting and/or determining the level of the target miRNA in the sample by contacting the sample with a probe that is specific for the target miRNA.

The sample may be any appropriate fluid or tissue sample obtained from the subject, as defined above. In some embodiments, the sample is blood, serum, plasma, urine, or saliva.

In some embodiments, the method may comprise determining the level of two or more target miRNAs in the sample.

According to a further aspect of the invention, there is provided a therapy for alleviating the musculoskeletal injury for use in a method of treating a subject in need thereof, wherein said subject is identified as having a musculoskeletal injury by determining a level of at least one miRNA in a sample from the subject.

The step of determining the level of the target miRNA may comprise contacting the sample with a substrate functionalized with the probe, for example a chip comprising the probe. The substrate or chip may conveniently include multiple probes, each specific for a different target miRNA.

The subject may have suffered an injury, in particular a musculoskeletal injury. The subject may be suspected as having a musculoskeletal injury. In some embodiments, the sample is obtained no more than 72 hours, no more than 48 hours, no more than 36 hours, no more than 24 hours, no more than 12 hours, no more than 6 hours, no more than 4 hours, no more than 2 hours or no more than 1 hour after injury. In some embodiments the sample is obtained 24 hours or more after the injury. In further embodiments the sample is obtained from the subject 15 days or fewer after injury. In some embodiments the sample is obtained from the subject between 24 hours and 15 days after injury, or between 24 hours and 10 days after injury, or between 24 hours and 7 days after injury, or between 48 hours and 5 days after injury.

The sensor element may further comprise a sample addition zone for receiving a sample (e.g. a fluid sample) thereon.

The probe is capable of selectively binding the miRNA of interest. The substrate may be functionalized with a plurality of probes. The probes may all be the same, or two or more different probes may be provided. For example, in some embodiments, the substrate may be functionalized with a first probe specific for a first miRNA, and a second probe specific for a second miRNA. The first and second probes may be grouped together, for example on different portions of the sensor element.

In a further aspect of the invention, there is provided a composition for use in a method of diagnosing and/or monitoring musculoskeletal injury in a subject, the composition comprising a probe specific for a target miRNA. The composition may comprise any one of the listed miRNAs or with any plurality of the listed miRNAs (e.g., two, three, four, or more of the listed miRNAs).

The probe may comprise a biological molecule such as a protein (e.g. an antibody) or a nucleic acid. In some embodiments, the probe comprises a nucleic acid. The nucleic acid may comprise a sequence which is at least 70%, at least 75%, at least 80%, at least 85%, at least 90% or at least 95% identical to a sequence which is the complement of the full-length sequence of the target miRNA. In some embodiments, the nucleic acid comprises a sequence which is 100% identical to a sequence which is the complement of the sequence of the target miRNA (i.e. the receptor comprises a nucleic acid sequence which is the exact complement of the target miRNA sequence).

The probes may be attached to a surface of the substrate by any suitable means, such as by coupling chemistry known to those skilled in the art. In some embodiments, each probe is attached to a surface of the substrate via a linker. In some embodiments, the probe comprises a moiety for immobilizing the probe on the substrate, or for attaching the probe to a linker immobilized on the substrate.

Alternatively or in addition, the probe may comprise a detectable label. The detectable label may be, for example, radioactive, fluorescent, luminescent, or antibody-based (e.g., it may constitute a conventional tetrameric antibody or a detectable fragment thereof).

The substrate of the sensor element may be formed from any suitable material. In some embodiments, the substrate comprises or is formed from metal, plastic, glass, silica, silicon, graphite, graphene, or any combination thereof. In some embodiments, the substrate comprises multiple layers. For example, a substrate may be prepared by forming a surface or layer of graphene on a layer of silicon carbide or silica. The graphene surface may be chemically modified, for example to graphene-oxide (GO) or graphene-amine (GA). Methods for forming graphene layers, such as epitaxial growth and sublimation growth, will be known to those skilled in the art.

Conveniently, probes comprising or constituted by a nucleic acid can be attached to a GO surface via a linker, using an amide coupling reagent (e.g. (O-(7-azabenzotriazole-1-yl)-N,N,N,N′-tetramethyluronium hexafluorophosphate (HATU)). A sensor element comprising a surface functionalized with a nucleic acid probe can then be used to selectively detect its complementary miRNA.

Suitable linkers may comprise an aniline moiety (or a derivative thereof), a benzoic acid moiety (or a derivative thereof) or an ethendiamine moiety (or a derivative thereof). An aniline linker may be formed by attaching a nitrobenzene molecule (or derivative) to a graphene surface (e.g. using a diazonium salt), and reducing the nitrobenzene to aniline. The amine group of the aniline may then be used to attach to the probe. Similarly, a diazonium salt (e.g. 4-benzoic acid diazonium tetrafluoroborate) can be used to attach a benzoic acid or benzoic acid derivative to a graphene surface. An ethanediamine moiety may be attached to carboxylated graphene or graphene oxide.

The sensor element may be comprised within a test strip. The test strip may be disposable.

The detection device may be configured to detect the binding of a target miRNA to the receptor by any suitable means known to those skilled in the art, for example by detecting changes in electrical impedance, hydrogen ion concentration, or conformational changes resulting from hybridization.

The detection device may further include a user interface to output data to a user.

In some embodiments, the detection device includes a database of treatment information. The device may be capable of identifying suitable treatment options from the database depending on the levels of the miRNA of interest. The treatment information may be provided to the user via the user interface.

Conveniently, the detection device may be portable, e.g. hand-held. The detection device may comprise a data storage unit for storing miRNA levels and other information relating to the subject. In some embodiments, the device comprises a data communication means for communicating data to other devices. For example, the device may communicate data wirelessly through WiFi, 3G, 4G, Bluetooth, or through a mobile app. This may conveniently enable the data to be easily accessed by medical professionals if necessary.

It is thus envisaged that the detection device of the invention provides an affordable, portable, point of care means for diagnosing and monitoring a muscolskeletal injury non-invasively. The device may be used by ambulance crews, the military, schools, sports clubs and healthcare professionals, enabling the correct assessment and triage of patients suspected to have a muscolskeletal injury.

A type of detection system is based on complementarity between a target miRNA and a nucleic acid probe, generally an oligonucleotide probe. The complementary or base pairing region can be 7 or 8 or more nucleotides in length. In embodiments the complementary or base pairing region can be 9, 10, 12, 15 or more nucleotides in length or the complementary or base pairing region can be the full length of the miRNA. The substrate functionalized with an oligonucleotide can be a bead or a nanoparticle. The detection system can have further components capable of converting bound nucleic acid probe-miRNA into a detectable signal.

Another type of detection system is based on RT-PCT. Therefore the present invention embraces a detection system for detecting and/or monitoring muscolskeletal injury in a subject comprising a primer pair designed for amplification of a cDNA complement of at least one miRNA described herein.

In a further aspect there is provided a kit for use in the present methods. The kit may comprise at least probe (e.g. a protein, such as an antibody, or a nucleic acid) which is capable of selectively binding the miRNA of interest. In some embodiments, the kit comprises an array comprising a plurality of probes. In some embodiments, the at least one probe is a primer for carrying out PCR. The kit may further comprise instructions for use, for example instructions for use in the diagnosis and/or monitoring of muscolskeletal injury. The kit may further comprise suitable buffers and reagents, such as amplification primers and enzymes (e.g. DNA polymerase, reverse transcriptase for conversion of miRNA to cDNA).

According to an aspect of the present disclosure, there is provided a method of diagnosing and treating a musculoskelatal injury in a human subject in need thereof, the method comprising: obtaining a body fluid sample from the subject; contacting the body fluid body fluid sample with a probe comprising a nucleic acid able to bind to at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, and put-miR-6, and Y_RNA.245; determining an amount of the at least one RNA biomarker in the body fluid samplebody fluid sample; identifying the subject as having a musculoskelatal injury where the amount of the at least one RNA biomarker is increased or decreased relative to a predetermined threshold value or relative to the amount of the RNA biomarker in a control sample; and treating the subject identified as having musculoskelatal injury.

In some embodiments, the body fluid sample is obtained during a period of time after injury selected from immediately after the injury to any period post-injury.

In some embodiments, the body fluid sample is obtained during a period of time after injury selected from immediately after the injury until the expression level of at least one of the RNA biomarkers returns to the predetermined threshold value or to the amount of the RNA biomarker in the control sample.

In some embodiments, the body fluid sample is obtained during a period of time after injury selected from immediately after the injury to up to 1 year, up to 10 months, up to 8 months, up to 6 months, up to 5 months, up to 4 months, up to 3 months, up to 2 months, up to 1 month, up to 25 days, up to 20 days, up to 15 days, up to 7 days, up to 5 days, up to 3 days, up to 2 days, and/or up to 24 hours post-injury.

In some embodiments, the body fluid sample is obtained during a period of time after injury selected from immediately after the injury to 15 days, from 1 hour to 15 days, from 24 hours to 15 days, from 24 hours to 7 days, and from 2 to 5 days.

In some embodiments, the method further comprises obtaining one or more additional body fluid samples from the subject at one or more additional times after the injury and repeating the detecting and amplifying steps for each additional sample.

In some embodiments, the one or more additional body fluid sample is obtained during a period of time after injury selected from immediately after the injury to any period post-injury.

In some embodiments, the one or more additional body fluid sample is obtained during a period of time after injury selected from immediately after the injury until the expression level of at least one of the RNA biomarkers returns to the predetermined threshold value or to the amount of the RNA biomarker in the control sample.

In some embodiments, the one or more additional body fluid sample is obtained during a period of time after injury selected from immediately after the injury to up to 1 year, up to 10 months, up to 8 months, up to 6 months, up to 5 months, up to 4 months, up to 3 months, up to 2 months, up to 1 month, up to 25 days, up to 20 days, up to 15 days, up to 7 days, up to 5 days, up to 3 days, up to 2 days, and/or up to 24 hours post-injury.

In some embodiments, the one or more additional body fluid sample is obtained during a period of time after injury selected from immediately after the injury to 15 days, from 1 hour to 15 days, from 24 hours to 15 days, from 24 hours to 7 days, and from 2 to 5 days.

In some embodiments, the one or more additional body fluid samples is obtained at day 1, 1.5, 2, 3, 4, 5, 6, 7, 8, 9, 10, 11, 12, 13, 14, or 15 after the injury.

In some embodiments, the detecting an amount of the at least one RNA biomarker is performed using a PCR-based assay, a light array assay, a laminar flow chip assay, or any assay suitable for detecting that at least one RNA biomarker.

In some embodiments, when using the PCR-based assay the predetermined threshold is equivalent to a fold change of 1.5 or more using the 2-delta delta CT (2-ΔΔCT) method.

In some embodiments, the predetermined threshold is equivalent to a fold change of 2 or more using the 2-delta delta CT (2-ΔΔCT) method.

In another aspect of the present disclosure, there is provided a method of diagnosing and/or monitoring musculoskelatal injury in a subject, the method comprising determining a level of at least one RNA biomarker in a body fluid sample obtained from the subject, wherein the at least one RNA biomarker is selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof.

In some embodiments, either an upregulated level or a downregulated level of the at least one RNA biomarker is indicative of a musculoskelatal injury.

In some embodiments, the subject is diagnosed as having a musculoskelatal injury if the level of the at least one RNA biomarker is either above or below a predetermined threshold or increased or decreased relative to a control.

In some embodiments, the method further comprises identifying the human subject as being fit for normal activity after undergoing successful treatment for a musculoskelatal injury.

In another aspect of the present disclosure, there is provided a sensor element for a detection system for diagnosing and/or monitoring a musculoskelatal injury, the sensor element comprising a substrate functionalized with a probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

In another aspect of the present disclosure, there is provided a detection system for diagnosing and/or monitoring a musculoskelatal injury, comprising a sensor element according to the present disclosure, and a detection device capable of detecting the binding of a target RNA biomarker to the probe.

In some embodiments, the detection system further comprises means to determine whether the target RNA biomarker is upregulated or downregulated.

In another aspect of the present disclosure, there is provided a method for determining a course of action for a subject suspected of having a musculoskelatal injury, comprising applying a body fluid sample obtained from the subject to a detection system according to the present disclosure, and if an upregulated level or a downregulated level of the at least one RNA biomarker is detected, providing a treatment for the musculoskelatal injury.

In another aspect of the present disclosure, there is provided a method of treating a subject with suspected of having a musculoskelatal injury, the method comprising determining whether an upregulated level or a downregulated level of at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245 is detectable in a body fluid sample obtained from the subject, and if an upregulated level or a downregulated level of at least one RNA biomarker is detected, providing treatment for the musculoskelatal injury to the subject.

In another aspect of the present disclosure, there is provided a method of detecting an RNA biomarker in a body fluid sample, the method comprising obtaining a body fluid sample from a human subject, contacting the body fluid sample with at least one oligonucleotide primer complementary to at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245; amplifying the at least one RNA biomarker using a polymerase chain reaction; and detecting the amplified RNA biomarker.

In another aspect of the present disclosure, there is provided a kit for use in a method of diagnosing and/or monitoring musculoskelatal injury in body fluid sample from a human subject, the kit comprising at least one probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

In another aspect of the present disclosure, there is provided a composition for use in a method of diagnosing and/or monitoring a musculoskelatal injury in a body fluid sample from a human subject, the composition comprising at least one probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

It will be appreciated that statements made herein in relation to any aspect of the invention may equally apply to any other aspect of the invention, as appropriate. Furthermore, the various aspects and embodiments of the present disclosure can also be used in combination with various techniques, methods, systems, compositions, devices, and/or disclosures as described in WO2017/153710A1 and WO2018/138468A1, the entire disclosures of which are hereby incorporated by reference herein.

The sequences and accession numbers for certain RNA biomarkers for musculoskeletal injury are provided in Table A below:

TABLE A

| | | miRBase | SEQ ID |
|---|---|---|---|
| RNA Biomarker | Sequence | Accession No. | NO. |
| put-miR-444 | CTGAAAAGGGGACGGATTGGGAA | | 1 |
| miR-143-3p | TGAGATGAAGCACTGTAGCTC | MIMAT0000435 | 2 |
| miR-34b-3p | CAATCACTAACTCCACTGCCAT | MIMAT0004676 | 3 |
| YRNA-684 | GCTTCTTTTACTCTTTCCCTTCATTCTCACTAC | | 4 |
| | TGTACCTGATTCGTCTT | | |
| put-miR-323 | ATAAAATGGGCGTTGAGG | | 5 |
| put-miR-893 | ACCATCCTCTGCTACCA | | 6 |
| miR-103a-3p | AGCAGCAUUGUACAGGGCUAUGA | MIMAT0000101 | 7 |
| YRNA-255 | GUGUCACCAACGUUGGUAUACAACCCCCCACAA | | 8 |
| | CUAAAUUUGACUGGCUU | | |
| UC022CJG1 | CATTGATCATCGACACTTCGAACGCACTTG | | 9 |
| put-miR-325 | TTCAAATCCCACTTCTGACACCA | | 10 |
| put-miR-469 | GCGGGGGATTAGCTCAGCTGGG | | 11 |
| put-miR-961 | TAGCTTGATCCAGTTG | | 12 |
| put-miR-476 | GTGGTCAGGTAGAGAA | | 13 |
| tRNA8-ThrAGT | GCGCCTGTCTAGTAAACAGGAGATCCTGGGTTC | | 14 |
| | GAATCCCAGCGGTGCCT | | |
| U2.3 | TCACTTCACGCATCGATCTGGTATTGCAGTACC | | 15 |
| | TCCAGGAACAGTGCACC | | |
| miR-148a-3p | UCAGUGCACUACAGAACUUUGU | MIMAT0000243 | 16 |
| miR-497-5p | CAGCAGCACACUGUGGUUUGU | MIMAT0002820 | 17 |
| miR-6748-3p | UCCUGUCCCUGUCUCCUACAG | MIMAT0027397 | 18 |
| put-miR-465 | CCAGTTGTCGTGGGTTTTT | | 19 |
| put-miR-6 | GCGGACCTTGCTCAAGG | | 20 |
| Y_RNA.245 | GTCTTTGTTGAACTCTTTCCCTCCTTCTCATTA | | 21 |
| | CTGTACTTGACCAGTCT | | |
| let-7a-5p | UGAGGUAGUAGGUUGUAUAGUU | MIMAT0000062 | 22 |
| let-7f-5p | UGAGGUAGUAGAUUGUAUAGUU | MIMAT0000067 | 23 |
| miR-135b-5p | UAUGGCUUUUCAUUCCUAUGUGA | MIMAT0000758 | 24 |
| miR-21-5p | UAGCUUAUCAGACUGAUGUUGA | MIMAT0000076 | 25 |
| miR-425-5p | AAUGACACGAUCACUCCCGUUGA | MIMAT0003393 | 26 |
| tRNA18ArgCCT | GCACTGGCCTCCTAAGCCAGGGATTGTGGGTTC | | 27 |
| | GAGTCCCACCTGGGGTA | | |
| tRNA120-AlaAGC | GCGCATGCTTAGCATGCATGAGGTCCCGGGTTC | | 28 |
| | GATCCCCAGCATCTCCA | | |
| tRNA73-ArgCCG | GCGTCTGATTCCGGATCAGAAGATTGAGGGTTC | | 29 |
| | GAGTCCCTTCGTGGTCG | | |
| miR-16-1-3p | CCAGUAUUAACUGUGCUGCUGA | MIMAT0004489 | 30 |
| miR-671-3p | UCCGGUUCUCAGGGCUCCACC | MIMAT0004819 | 31 |
| put-miR-1306 | ATAACGTCATCTAGTGTG | | 32 |
| SNORA57 | TGCTGGCGGCTTCCCATCCGCTGGTTCTATCCT | | 33 |
| | CAAACGCCGGGACACCG | | |
| tRNA27-MetCAT | GCGTCAGTCTCATAATCTGAAGGTCCTGAGTTC | | 34 |
| | GAGCCTCAGAGAGGGCA | | |
| tRNA8-ThrAGT | GCGCCTGTCTAGTAAACAGGAGATCCTGGGTTC | | 35 |
| | GAATCCCAGCGGTGCCT | | |
| miR-107 | AGCAGCAUUGUACAGGGCUAUCA | MIMAT0000104 | 36 |
| miR-1246 | AAUGGAUUUUUGGAGCAGG | MIMAT0005898 | 37 |
| miR-148a-3p | UCAGUGCACUACAGAACUUUGU | MIMAT0000243 | 38 |
| miR-339-5p | UCCCUGUCCUCCAGGAGCUCACG | MIMAT0000764 | 39 |
| U6.1249 | GATGGCATGACCCCTGATCAAGGACGGCATGCA | | 40 |
| | AATTTGTGAAGTATTTC | | |
| miR-126-3p | UCGUACCGUGAGUAAUAAUGCG | MIMAT0000445 | 41 |
| tRNA84-GluTTC | TTTCACCGCCGCGGCCCGGGTTCGATTCCCGGT | | 42 |
| | CAGGGAA | | |
| miR-142-3p | UGUAGUGUUUCCUACUUUAUGGA | MIMAT0000434 | 43 |
| miR-142-5p | CAUAAAGUAGAAAGCACUACU | MIMAT0000433 | 44 |
| put-miR-188 | AAATGGCGATACTCAGG | | 45 |
| SNORD3B-2 | CTTCTCTCCGTATTGGGGAGTGAGAGGGAGAGA | | 46 |
| | ACGCGGTCTGAGTGGTT | | |
| U6.168 | ATGGCCCCTGCGCAAGGATGACACGCAAATTTG | | 47 |
| | TGAAGGATTCCATATTT | | |

As discussed in more detail in Example 1 (below), these biomarkers were found to be differentially expressed and statistically significant between the different group comparisons and the different time points (MSK vs U at timepoint b, MSK vs B at timepoint b, MSK vs U at timepoint c, MSK vs B at timepoint c).

According to an aspect of the present disclosure, there is provided a method of diagnosing and treating a musculoskelatal injury in a human subject in need thereof, the method comprising: obtaining a body fluid sample from the subject; contacting the body fluid sample with a probe comprising a nucleic acid able to bind to at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245 determining an amount of the at least one RNA biomarker in the body fluid sample; identifying the subject as having a musculoskelatal injury where the amount of the at least one RNA biomarker is increased or decreased relative to a predetermined threshold value or relative to the amount of the RNA biomarker in a control sample; and treating the subject identified as having the musculoskelatal injury by administering to the subject one or more therapies.

In some embodiments, the method further comprises identifying the human subject as being fit for normal activity after undergoing successful treatment for a musculoskelatal injury.

In another aspect of the present disclosure, there is provided a method of diagnosing and/or monitoring a musculoskelatal injury in a subject, the method comprising determining a level of at least one RNA biomarker in a body fluid sample obtained from the subject, wherein the at least one RNA biomarker is selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof.

In some embodiments, either an upregulated level or a downregulated level of the at least one RNA biomarker is indicative of a musculoskelatal injury.

In some embodiments, the subject is diagnosed as having TBI if the level of the at least one RNA biomarker is either above or below a predetermined threshold or increased or decreased relative to a control.

In some embodiments, the method further comprises identifying the human subject as being fit for normal activity after undergoing successful treatment for a musculoskelatal injury.

In another aspect of the present disclosure, there is provided a sensor element for a detection system for diagnosing and/or monitoring a musculoskelatal injury, the sensor element comprising a substrate functionalized with a probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

In another aspect of the present disclosure, there is provided a detection system for diagnosing and/or monitoring a musculoskelatal injury, comprising a sensor element according to the present disclosure, and a detection device capable of detecting the binding of a target RNA biomarker to the probe.

In some embodiments, the detection system further comprises means to determine whether the target RNA biomarker is upregulated or downregulated.

In another aspect of the present disclosure, there is provided a method for determining a course of action for a subject suspected of having a musculoskelatal injury, comprising applying a body fluid sample obtained from the subject to a detection system according to the present disclosure, and if an upregulated level or a downregulated level of the at least one RNA biomarker is detected, providing a treatment for the musculoskelatal injury.

In another aspect of the present disclosure, there is provided a method of treating a subject with suspected of having a musculoskelatal injury, the method comprising determining whether an upregulated level or a downregulated level of at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245 is detectable in a body fluid sample obtained from the subject, and if an upregulated level or a downregulated level of at least one RNA biomarker is detected, providing treatment for the musculoskelatal injury to the subject.

In another aspect of the present disclosure, there is provided a method of detecting an RNA biomarker in a body fluid sample, the method comprising obtaining a body fluid sample from a human subject, contacting the body fluid sample with at least one oligonucleotide primer complementary to at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245; amplifying the at least one RNA biomarker using a polymerase chain reaction; and detecting the amplified RNA biomarker.

In another aspect of the present disclosure, there is provided a kit for use in a method of diagnosing and/or monitoring a musculoskelatal injury in a body fluid sample from a human subject, the kit comprising at least one probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

In another aspect of the present disclosure, there is provided a composition for use in a method of diagnosing and/or monitoring a musculoskelatal injury in a body fluid sample from a human subject, the composition comprising at least one probe specific for at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245.

In some embodiments, the probe comprises a nucleic acid able to bind to the at least one RNA biomarker.

In some embodiments, the probe comprises a nucleic acid having at least 70% identity with a sequence which is the complement of the sequence of the target RNA biomarker.

In some embodiments, the probe comprises a nucleic acid having at least 70% identity with a sequence which is the complement of SEQ ID NOs: 1-21 and/or SEQ ID NOs: 22-47.

It will be appreciated that statements made herein in relation to any aspect of the invention may equally apply to any other aspect of the invention, as appropriate.

### EXAMPLES

Embodiments of the invention will now be described by way of example.

### Example 1

### Musculoskelatal Injury Biomarker Screening

### Study Approval

The Study of Concussion in Rugby Union through MicroRNAs (SCRUM) was conducted as part of the ReCoS (The REpetitive COncussion in Sport). This study was carried out in accordance with the requirements of the University of Birmingham Research Ethics Committee who approved the study in addition to the East of England NHS Ethics Committee (Ref. 11-0429AP28). All subjects gave written informed consent in accordance with the Declaration of Helsinki.

### Study Design

The study was pre-registered with the International Standard Randomised Controlled Trials Number in February 2018 (ISRCTN16974791). The full study methodology, including the analysis plan, was published in 2018 (Yakoub K M, O'Halloran P, Davies D J, et al. Study of Concussion in Rugby Union through MicroRNAs (SCRUM): a study protocol of a prospective, observational cohort study. BMJ Open 2018; 8(11): e024245.).

Briefly, 2 ml of saliva was collected at enrolment (pre-season) from all players participating to the study (this group was defined as baseline or B group) and from all players undergoing concussion assessment each time they entered the Head Injury Assessment (HIA) process. This includes players that were immediately and permanently removed due to incontrovertible signs of concussion, as well as players removed following the pitch-side assessment (time point a). All players with confirmed or suspected concussion (group C) provided a body fluid sample post-match (time point b) and at 36 h-48 h (time point c).

Every time a player was assessed for concussion, clubs were also ask to identify another player who had played a similar number of minutes and in a similar position to serve as uninjured control (group U). If, during the same match, somebody was removed due to a musculoskeletal injury, the clubs were asked to approach the player for a body fluid sample (group MSK).

### Saliva Collection

Medical staff at the respective clubs were trained in the collection procedure. Saliva was collected in Oragene®-RNA RE-100 saliva self-collection kits (DNA Genotek) containing an RNA stabilizing solution preserving the samples for up to 8 weeks. Following an oral rinse with tap water, saliva was collected from each participant at enrolment and at the different time points after injury as described above. Samples were transported to the University of Birmingham, where they were processed in line with the manufacturer's protocol for storage.

### RNA Preparation

RNA preparation was performed according to DNA Genotek recommendations at QIAGEN Genomic Services, Germany, with the use of the miRNeasy extraction kits (Qiagen, Germany).

### Next Generation Sequencing (NGS)—Discovery Phase

Small non-coding RNAs (sncRNAs) Next Generation Sequencing (NGS) was performed in the initial discovery phase using 15 saliva baseline samples (B), 15 samples from concussed players (C) and 20 controls (consisting of 10 MSK and 10 U players). All samples analyzed in this phase were collected post-match (time point b).

### Library Preparation and Next Generation Sequencing

Library preparation was carried out using the QIAseq miRNA Library Kit (QIAGEN). A total of 5 ul total RNA was converted into microRNA NGS libraries. Adapters containing UMIs were ligated to the RNA. Then RNA was converted to cDNA. The cDNA as amplified using PCR (22 cycles) and during the PCR, indices were added. After PCR the samples were purified. Library preparation QC was performed using either the Bioanalyzer 2100 (Agilent) or TapeStation4200 (Agilent). Based on quality of the inserts and the concentration measurements the libraries were pooled in equimolar ratios. The library pool(s) were quantified using the qPCR ExiSEQ LNA™ Quant kit (Exiqon). The library pools were then sequenced on a NextSeq500 sequencing instrument according to the manufacturers instructions (NEBNext Multiplex Small RNA Library Prep Set for Illumina) to make approximately 163-175 base-pair sized libraries. Raw data as demultiplexed and FASTQ files for each sample were generated using the bcl2fastq software (Illumina inc.). FASTQ data were checked using the FastQC tool (bioinformatics.babraham.ac.uk/proeects/fastqc/).

### Mapping

A reference profile of sequencing data for each sample was obtained using the whole human genome sequence GRCh37, downloaded from the Genome Reference Consortium and mirbase_20 as an annotation reference. Reads were aligned to the miRbase using Bowtie2. The mapping criteria for aligning reads to spike-ins, abundant sequence and miRBase were the reads to have perfect match to the reference sequences. For mapping to the genome, the restricting was one mismatch which was allowed in the first 32 bases of the read. No in-dels were allowed in mapping. Unaligned reads were mapped against the host reference genome and used as input for mirPara and miRbase to predict putative miRNAs.

### Statistical Analysis

Aligned reads were counted and differential expression analysis, p-values for significantly differentially expressed microRNAs and false discovery rate according to Benjamini-Hochberg were performed with EdgeR. For normalisation, the trimmed mean of M-values (TMM) method based on log-fold and absolute gene-wise changes in expression levels between samples was used.

MiRNA qPCR Data Analysis—Validation Study

All laboratory analyses were conducted at QIAGEN Genomic Services, Germany.

MiRNA qPCR validation was performed in 2 steps in a total of 176 saliva baseline samples (B); 43 samples form concussed players after injury (group HIA+ a); 53 samples from concussed players post-match (group HIA+ b) and 55 concussed players after 36-48 h (group HIA+ c); 33 HIA− players after injury (group HIA− a); 25 HIA− players post-match (group HIA− b); 20 HIA− players after 36-48 h (group HIA− c); 62 U players post-match (group Ub); 46 U players at 36-48 h (group Uc); 31 MSK players post-match (group MSK b) and 25 MSK players at 36-48 h (group MSK c).

qPCR

14 μl RNA was reverse transcribed in 70 μl reactions using the miRCURY LNA RT Kit (QIAGEN). cDNA was diluted 50× and assayed in 10 μl PCR reactions according to the protocol for miRCURY LNA miRNA PCR; each miRNA was assayed once by qPCR on the miRNA Ready-to-Use PCR, custom panel using miRCURY LNA SYBR Green master mix. Negative controls excluding template from the reverse transcription reaction was performed and profiled like the samples. The amplification was performed in a LightCyclerp 480 Real-Time PCR System (Roche) in 384 well plates. The amplification curves were analysed using the Roche LC software, both for determination of Cq (by the 2nd derivative method) and for melt curve analysis. The amplification efficiency was calculated using algorithms similar to the LinReg software. All assays were inspected for distinct melting curves (Tm) and the Tm was checked to be within known specifications for the assay. Furthermore, assays must be detected with 0 Cq less than the negative control, and with Cq<37 to be included in the data analysis. Data that did not pass these criteria were omitted from any further analysis. Cq was calculated as the 2nd derivative. Normalization was performed based on the average of hsa-miR-29c-3p and hsa-let-7b-5p (custom normalizer assays), the two most stable miRs identified across all samples by Normofinder software (36). The formula used to calculate the normalized Cq values is the difference between the custom normalizer assays mean Cq and the assay Cq (miRNA of interest). After normalization 20 has been added to the normalized dCq values to shift the numbers in a positive range to allow using the qPCR analysis pipelins according Qiagen procedures. While processing the data in the qPCR pipeline a minus is inserted before the normalized dCq value. A higher value indicates that the miRNA is more abundant in that sample.

### Statistical Analysis

Normalized dCq values were checked to be normally distributed and then used to perform the two-tailed Independent-Samples t test procedure in order to compare means of pair groups, MSK group was compared to B and to U group at time point b and c respectively.

Results are presented in Table B below. MSK refers to the group identified with a musculoskeletal injury. U refers to the uninjured group. B refers to the baseline group. Time point b is post-match. Time point c is 36-48 hours after the match.

TABLE B

| MSK vs U | MSK vs U | MSK vs B | MSK vs B |
|---|---|---|---|
| Time Point b | Time Point c | Time Point b | Time Point c |
| (Post-Match) | (36-48 h)) | (Post-Match) | (36-48 h)) |
| | | let-7a-5p | let-7a-5p |
| let-7f-5p | let-7f-5p | let-7f-5p | let-7f-5p |
| | miR-135b-5p | miR-135b-5p | miR-135b-5p |
| | miR-21-5p | miR-21-5p | miR-21-5p |
| | miR-425-5p | miR-425-5p | miR-425-5p |
| | put-miR-444 | | |
| | tRNA18ArgCCT | | |
| | miR-143-3p | | |
| miR-34b-3p | | | |
| tRNA120- | tRNA120- | | tRNA120- |
| AlaAGC | AlaAGC | | AlaAGC |
| | tRNA73-ArgCCG | | |
| | YRNA-684 | | YRNA-684 |
| miR-16-1-3p | miR-16-1-3p | miR-16-1-3p | miR-16-1-3p |
| put-miR-323 | | | |
| | put-miR-893 | put-miR-893 | put-miR-893 |
| miR-103a-3p | miR-103a-3p | miR-103a-3p | miR-103a-3p |
| | miR-671-3p | miR-671-3p | |
| put-miR-1306 | | | |
| | YRNA-255 | | |
| | SNORA57 | SNORA57 | SNORA57 |
| | UC022CJG1 | | |
| | tRNA27-MetCAT | | tRNA27-MetCAT |
| | tRNA8-ThrAGT | | tRNA8-ThrAGT |
| | put-miR-325 | | put-miR-325 |
| | | miR-107 | miR-107 |
| | | | miR-1246 |
| | | | miR-148a-3p |
| | | miR-339-5p | miR-339-5p |
| | | | put-miR-469 |
| | put-miR-961 | | |
| | U6.1249 | | |
| | put-miR-476 | | |
| miR-126-3p | | | |
| | | | tRNA84-GluTTC |
| | | tRNA8-ThrAGT | tRNA8-ThrAGT |
| | | | U2.3 |
| | | miR-142-3p | |
| | | miR-142-5p | |
| | | miR-148a-3p | |
| | | miR-497-5p | |
| | | miR-6748-3p | |
| | | put-miR-188 | |
| | | put-miR-465 | |
| | | put-miR-6 | |
| | | SNORD3B-2 | |
| | | U6.168 | |
| | | Y_RNA.245 | |

Other advantages that are obvious and/or inherent to the disclosure will be evident to one skilled in the art. It will be understood that certain features and sub-combinations are of utility and may be employed without reference to other features and sub-combinations. This is contemplated by and is within the scope of the claims. Since many possible embodiments may be made of the disclosure without departing from the scope thereof, it is to be understood that all matter herein set forth or shown in the accompanying drawings is to be interpreted as illustrative and not in a limiting sense.

1. A method of diagnosing and/or monitoring a musculoskelatal injury in a subject, the method comprising:
determining a level of at least one RNA biomarker in a body fluid sample obtained from the subject, wherein the at least one RNA biomarker is selected from the group consisting of put-miR-444, put-miR-323, put-miR-469, put-miR-961, miR-6748-3p, and put-miR-465, or any combination thereof;
wherein the subject is diagnosed as having a musculoskelatal injury if the level of the at least one RNA biomarker is either above or below a predetermined threshold or increased or decreased relative to a control.

2. The method of claim 1 further comprising:
determining a level of at least one additional RNA biomarker selected from the group consisting of miR-143-3p, miR-34b-3p, YRNA-684, put-miR-893, miR-103a-3p, YRNA-255, UC022CJG1, put-miR-325, put-miR-476, tRNA8-ThrAGT, U2.3, miR-148a-3p, miR-497-5p, put-miR-6, and Y_RNA.245, or any combination thereof;
wherein the subject is diagnosed as having a musculoskelatal injury if the level of the at least one additional RNA biomarker is either above or below a predetermined threshold or increased or decreased relative to a control.

3. The method of claim 1, further comprising:
determining a level of at least one more RNA biomarker selected from the group consisting of let-7a-5p, let-7f-5p, miR-135b-5p, miR-21-5p, miR-425-5p, tRNA18ArgCCT, tRNA120-AlaAGC, tRNA73-ArgCCG, miR-16-1-3p, miR-671-3p, put-miR-1306, SNORA57, tRNA27-MetCAT, tRNA8-ThrAGT, miR-107, miR-1246, miR-148a-3p, miR-339-5p, U6.1249, miR-126-3p, tRNA84-GluTTC, miR-142-3p, miR-142-5p, put-miR-188, SNORD3B-2, AND U6.168, or any combination thereof;
wherein the subject is diagnosed as having a musculoskelatal injury if the level of the at least more RNA biomarker is either above or below a predetermined threshold or increased or decreased relative to a control.

4. The method of claim 1, wherein the body fluid sample is obtained during a period of time after injury selected from immediately after the injury to one year post-injury.

5. The method of claim 4, wherein the body fluid sample is obtained during a period of time after injury selected from immediately after the injury until the expression level of at least one of the RNA biomarkers returns to the predetermined threshold value or to the amount of the RNA biomarker in the control sample.

6. The method of claim 4, wherein the body fluid sample is obtained during a period of time after injury selected from immediately after the injury to up to 1 year, up to 10 months, up to 8 months, up to 6 months, up to 5 months, up to 4 months, up to 3 months, up to 2 months, up to 1 month, up to 25 days, up to 20 days, up to 15 days, up to 7 days, up to 5 days, up to 3 days, up to 2 days, and/or up to 24 hours post-injury.

7. The method of claim 6, wherein the body fluid sample is obtained during a period of time after injury selected from immediately after the injury to 15 days, from 1 hour to 15 days, from 24 hours to 15 days, from 24 hours to 7 days, and from 2 to 5 days.

8. The method of claim 1, further comprising obtaining one or more additional body fluid samples from the subject at one or more additional times after the injury and repeating the detecting step for each additional sample.

9. The method of claim 8, wherein the one or more additional body fluid sample is obtained during a period of time after injury selected from immediately after the injury until the expression level of at least one of the RNA biomarkers returns to the predetermined threshold value or to the amount of the RNA biomarker in the control sample.

10. The method of claim 8, wherein the one or more additional body fluid sample is obtained during a period of time after injury selected from immediately after the injury to up to 1 year, up to 10 months, up to 8 months, up to 6 months, up to 5 months, up to 4 months, up to 3 months, up to 2 months, up to 1 month, up to 25 days, up to 20 days, up to 15 days, up to 7 days, up to 5 days, up to 3 days, up to 2 days, and/or up to 24 hours post-injury.

11. The method of claim 8, wherein the one or more additional body fluid sample is obtained during a period of time after injury selected from immediately after the injury to 15 days, from 1 hour to 15 days, from 24 hours to 15 days, from 24 hours to 7 days, and from 2 to 5 days.

12. The method of claim 8, wherein the one or more additional body fluid samples is obtained between day 1 and day 15 after the injury.

13. The method of claim 1, wherein the detecting an amount of the at least one RNA biomarker is performed using a PCR-based assay, a light array assay, a laminar flow chip assay, or any assay suitable for detecting that at least one RNA biomarker.

14. The method of claim 13, wherein when using the PCR-based assay the predetermined threshold is equivalent to a fold change of 1.5 or more using the 2-delta delta CT (2-ΔΔCT) method.

15. The method of claim 14, wherein the predetermined threshold is equivalent to a fold change of 2 or more using the 2-delta delta CT (2-ΔΔCT) method.

16. The method according to claim 1, further comprising identifying the human subject as being fit for normal activity after undergoing successful treatment for a musculoskelatal injury.

17-21. (canceled)

22. A detection system for diagnosing and/or monitoring a musculoskelatal injury, comprising:
a sensor element comprising a substrate functionalized with a probe specific for at least one RNA biomarker selected from the group consisting of put-miR-444, put-miR-323, put-miR-469, put-miR-961, miR-6748-3p, and put-miR-465, or any combination thereof; and
a detection device capable of detecting the binding of a target RNA biomarker to the probe.

23. (canceled)

24. A method for determining a course of action for a subject suspected of having a musculoskeletal injury, comprising applying a body fluid sample obtained from the subject to a detection system according to claim 22, and if an upregulated level or a downregulated level of the at least one RNA biomarker is detected, providing a treatment for a musculoskelatal injury.

25. A method of treating a subject suspected of having a musculoskeletal injury, the method comprising
determining whether an upregulated level or a downregulated level of at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof; and/or is detectable in a body fluid sample obtained from the subject;
diagnosing the subject as having a musculoskeletal injury if an upregulated level or a downregulated level of at least one RNA biomarker is detected; and
providing treatment for the musculoskeletal injury to the subject.

26-38. (canceled)

39. A method of diagnosing and treating a musculoskeletal injury in a human subject in need thereof, the method comprising:
obtaining a body fluid sample from the subject;
contacting the body fluid sample with a probe comprising a nucleic acid able to bind to at least one RNA biomarker selected from the group consisting of put-miR-444, miR-143-3p, miR-34b-3p, YRNA-684, put-miR-323, put-miR-893, miR-103a-3p, YRNA-255, UC022CJG1, put-miR-325, put-miR-469, put-miR-961, put-miR-476, tRNA8-ThrAGT, U2.3, miR-148a-3p, miR-497-5p, miR-6748-3p, put-miR-465, put-miR-6, and Y_RNA.245, or any combination thereof;
determining an amount of the at least one RNA biomarker in the body fluid sample;
identifying the subject as having a musculoskelatal injury where the amount of the at least one RNA biomarker is increased or decreased relative to a predetermined threshold value or relative to the amount of the RNA biomarker in a control sample; and
treating the subject identified as having a musculoskelatal injury by administering to the subject one or more therapies.

40-51. (canceled)


SEQUENCE LISTING (47 sequences)

SEQ ID NO: 1 - DNA, 23 bp, Homo sapiens
  source 1..23: mol_type = genomic DNA
ctgaaaaggggacggattgggaa

SEQ ID NO: 2 - DNA, 21 bp, Homo sapiens
  source 1..21: mol_type = genomic DNA
tgagatgaagcactgtagctc

SEQ ID NO: 3 - DNA, 22 bp, Homo sapiens
  source 1..22: mol_type = genomic DNA
caatcactaactccactgccat

SEQ ID NO: 4 - DNA, 50 bp, Homo sapiens
  source 1..50: mol_type = genomic DNA
gcttcttttactctttcccttcattctcactactgtacctgattcgtctt

SEQ ID NO: 5 - DNA, 18 bp, Homo sapiens
  source 1..18: mol_type = genomic DNA
ataaaatgggcgttgagg

SEQ ID NO: 6 - DNA, 17 bp, Homo sapiens
  source 1..17: mol_type = genomic DNA
accatcctctgctacca

SEQ ID NO: 7 - RNA, 23 nt, Homo sapiens
  source 1..23: mol_type = genomic RNA
agcagcattgtacagggctatga

SEQ ID NO: 8 - RNA, 50 nt, Homo sapiens
  source 1..50: mol_type = genomic RNA
gtgtcaccaacgttggtatacaaccccccacaactaaatttgactggctt

SEQ ID NO: 9 - DNA, 30 bp, Homo sapiens
  source 1..30: mol_type = genomic DNA
cattgatcatcgacacttcgaacgcacttg

SEQ ID NO: 10 - DNA, 23 bp, Homo sapiens
  source 1..23: mol_type = genomic DNA
ttcaaatcccacttctgacacca

SEQ ID NO: 11 - DNA, 22 bp, Homo sapiens
  source 1..22: mol_type = genomic DNA
gcgggggattagctcagctggg

SEQ ID NO: 12 - DNA, 16 bp, Homo sapiens
  source 1..16: mol_type = genomic DNA
tagcttgatccagttg

SEQ ID NO: 13 - DNA, 16 bp, Homo sapiens
  source 1..16: mol_type = genomic DNA
gtggtcaggtagagaa

SEQ ID NO: 14 - DNA, 50 bp, Homo sapiens
  source 1..50: mol_type = genomic DNA
gcgcctgtctagtaaacaggagatcctgggttcgaatcccagcggtgcct

SEQ ID NO: 15 - DNA, 50 bp, Homo sapiens
  source 1..50: mol_type = genomic DNA
tcacttcacgcatcgatctggtattgcagtacctccaggaacagtgcacc

SEQ ID NO: 16 - RNA, 22 nt, Homo sapiens
  source 1..22: mol_type = genomic RNA
tcagtgcactacagaactttgt

SEQ ID NO: 17 - RNA, 21 nt, Homo sapiens
  source 1..21: mol_type = genomic RNA
cagcagcacactgtggtttgt

SEQ ID NO: 18 - RNA, 21 nt, Homo sapiens
  source 1..21: mol_type = genomic RNA
tcctgtccctgtctcctacag

SEQ ID NO: 19 - DNA, 19 bp, Homo sapiens
  source 1..19: mol_type = genomic DNA
ccagttgtcgtgggttttt

SEQ ID NO: 20 - DNA, 17 bp, Homo sapiens
  source 1..17: mol_type = genomic DNA
gcggaccttgctcaagg

SEQ ID NO: 21 - DNA, 50 bp, Homo sapiens
  source 1..50: mol_type = genomic DNA
gtctttgttgaactctttccctccttctcattactgtacttgaccagtct

SEQ ID NO: 22 - RNA, 22 nt, Homo sapiens
  source 1..22: mol_type = genomic RNA
tgaggtagtaggttgtatagtt

SEQ ID NO: 23 - RNA, 22 nt, Homo sapiens
  source 1..22: mol_type = genomic RNA
tgaggtagtagattgtatagtt

SEQ ID NO: 24 - RNA, 23 nt, Homo sapiens
  source 1..23: mol_type = genomic RNA
tatggcttttcattcctatgtga

SEQ ID NO: 25 - RNA, 22 nt, Homo sapiens
  source 1..22: mol_type = genomic RNA
tagcttatcagactgatgttga

SEQ ID NO: 26 - RNA, 23 nt, Homo sapiens
  source 1..23: mol_type = genomic RNA
aatgacacgatcactcccgttga

SEQ ID NO: 27 - DNA, 50 bp, Homo sapiens
  source 1..50: mol_type = genomic DNA
gcactggcctcctaagccagggattgtgggttcgagtcccacctggggta

SEQ ID NO: 28 - DNA, 50 bp, Homo sapiens
  source 1..50: mol_type = genomic DNA
gcgcatgcttagcatgcatgaggtcccgggttcgatccccagcatctcca

SEQ ID NO: 29 - DNA, 50 bp, Homo sapiens
  source 1..50: mol_type = genomic DNA
gcgtctgattccggatcagaagattgagggttcgagtcccttcgtggtcg

SEQ ID NO: 30 - RNA, 22 nt, Homo sapiens
  source 1..22: mol_type = genomic RNA
ccagtattaactgtgctgctga

SEQ ID NO: 31 - RNA, 21 nt, Homo sapiens
  source 1..21: mol_type = genomic RNA
tccggttctcagggctccacc

SEQ ID NO: 32 - DNA, 18 bp, Homo sapiens
  source 1..18: mol_type = genomic DNA
ataacgtcatctagtgtg

SEQ ID NO: 33 - DNA, 50 bp, Homo sapiens
  source 1..50: mol_type = genomic DNA
tgctggcggcttcccatccgctggttctatcctcaaacgccgggacaccg

SEQ ID NO: 34 - DNA, 50 bp, Homo sapiens
  source 1..50: mol_type = genomic DNA
gcgtcagtctcataatctgaaggtcctgagttcgagcctcagagagggca

SEQ ID NO: 35 - DNA, 50 bp, Homo sapiens; identical to SEQ ID NO: 14
  source 1..50: mol_type = genomic DNA

SEQ ID NO: 36 - RNA, 23 nt, Homo sapiens
  source 1..23: mol_type = genomic RNA
agcagcattgtacagggctatca

SEQ ID NO: 37 - RNA, 19 nt, Homo sapiens
  source 1..19: mol_type = genomic RNA
aatggatttttggagcagg

SEQ ID NO: 38 - RNA, 22 nt, Homo sapiens
  source 1..22: mol_type = genomic RNA
tcagtgcactacagaactttgt

SEQ ID NO: 39 - RNA, 23 nt, Homo sapiens
  source 1..23: mol_type = genomic RNA
tccctgtcctccaggagctcacg

SEQ ID NO: 40 - DNA, 50 bp, Homo sapiens
  source 1..50: mol_type = genomic DNA
gatggcatgacccctgatcaaggacggcatgcaaatttgtgaagtatttc

SEQ ID NO: 41 - RNA, 22 nt, Homo sapiens
  source 1..22: mol_type = genomic RNA
tcgtaccgtgagtaataatgcg

SEQ ID NO: 42 - DNA, 40 bp, Homo sapiens
  source 1..40: mol_type = genomic DNA
tttcaccgccgcggcccgggttcgattcccggtcagggaa

SEQ ID NO: 43 - RNA, 23 nt, Homo sapiens
  source 1..23: mol_type = genomic RNA
tgtagtgtttcctactttatgga

SEQ ID NO: 44 - RNA, 21 nt, Homo sapiens
  source 1..21: mol_type = genomic RNA
cataaagtagaaagcactact

SEQ ID NO: 45 - DNA, 17 bp, Homo sapiens
  source 1..17: mol_type = genomic DNA
aaatggcgatactcagg

SEQ ID NO: 46 - DNA, 50 bp, Homo sapiens
  source 1..50: mol_type = genomic DNA
cttctctccgtattggggagtgagagggagagaacgcggtctgagtggtt

SEQ ID NO: 47 - DNA, 50 bp, Homo sapiens
  source 1..50: mol_type = genomic DNA
atggcccctgcgcaaggatgacacgcaaatttgtgaaggattccatattt